(19) United States
(12) Patent Application Publication
(10) Pub. No.: US 2023/0382966 A1
(43) Pub. Date: Nov. 30, 2023

(54) NOVEL KLOTHO INTERACTION SITE IN THE C-TERMINUS OF FGF23

(71) Applicant: INDIANA UNIVERSITY RESEARCH AND TECHNOLOGY CORPORATION, Indianapolis, IN (US)

(72) Inventors: Richard D. DIMARCHI, Carmel, IN (US); Archita S. AGRAWAL, Bloomington, IN (US)

(21) Appl. No.: 18/032,479

(22) PCT Filed: Oct. 26, 2021
(86) PCT No.: PCT/US2021/056563

§ 371 Date: Apr. 18, 2023

Related U.S. Application Data
(60) Provisional application No. 63/105,965, filed on Oct. 27, 2020.

(51) Int. Cl. C07K 14/50

(52) U.S. Cl. CPC C07K 14/50; A61K 38/00

(57) ABSTRACT

Compositions comprising novel peptides and dimers that exhibit antagonist activity against FGF23 binding to Klotho are disclosed. Such peptides can be used to treat hypoparathyroidism and osteoporosis.

### CROSS REFERENCE TO RELATED APPLICATIONS

This application claims priority to U.S. Provisional Patent Application No. 63/105,965 filed on Oct. 27, 2020, the disclosure of which is expressly incorporated herein.

### INCORPORATION BY REFERENCE OF MATERIAL SUBMITTED ELECTRONICALLY

Incorporated by reference in its entirety is a computer-readable nucleotide/amino-acid sequence listing submitted concurrently herewith and identified as follows: 15 kilobytes ACII (text) file named “348560_ST25.txt,” created on Oct. 25, 2021.

### BACKGROUND

Fibroblast growth factor 23 (FGF23) belongs to the endocrine FGF-family of proteins. The FGF23 gene was discovered as the underlying cause for autosomal dominant hypophosphatemia rickets (ADHR) and tumor induced osteomalacia (TIO). Genetic mutations in FGF23 at select Arg^{176,179} residues were identified as the molecular basis for hypophosphatemia arising from sustained action of proteolytically resistant FGF23 proteins. Subsequently, it was identified that FGF23 regulates phosphate metabolism by its direct action on the kidney. It suppresses the function of the sodium phosphate co-transporter NaPi-2a (SLC34A1) in renal tubules to promote phosphate excretion. Simultaneously, FGF23 reduces circulating 1, 25-dihydroxy vitamin D₃ through regulation of the vitamin D metabolizing enzymes that suppress its synthesis and accelerate catabolism. These collective actions accelerate phosphate clearance and reduce serum phosphate levels.

At a cellular level, FGF23 activates signaling through a receptor complex of FGF-receptor (FGFR) and Klotho (KL). The N-terminus of FGF23 primarily interacts with the FGFR while the C-terminus engages KL. The receptor-interactions have been elucidated in the crystal structure of the ternary-receptor complex FGF23/FGFR/KL. The C-terminal truncation of FGF23 up to 48 amino acids did not alter its biological activity. Furthermore, competitive FGF23-antagonism with a C-terminally derived FGF23 fragment of 26-aa length (C26, FGF23¹⁸⁰⁻²⁰⁵), coupled with structural studies have demonstrated the direct association of this peptide fragment with KL. These results collectively show that a relatively short C-terminal FGF23 peptide supports the receptor interactions leading to downstream biological signaling.

The other endocrine FGFs members (FGF19 and FGF21) use FGFR in the presence of Klotho-β (KLB) to promote their biological action at liver, adipose and pancreas. Physiologically FGF19 regulates bile acid metabolism, while FGF21 controls the circulating levels of glucose and lipid. The critical interactions between the C-terminus of FGF19/FGF21 and their co-receptor KLB have been outlined in their respective crystal structures. Comparably-sized C-terminal peptides of FGF19 and FGF21 can competitively inhibit the activity of these proteins, similar to what is observed with FGF23 C26 and KL. These studies promote a biochemical basis of interaction between the endocrine FGFs and their receptor complexes to define a well-aligned C-terminal sequence of approximately 25-aa that engages with the respective co-receptors KL or KLB. However, it is a mystery as to why nature extended the C-terminus of FGF23 more than forty amino acids beyond the point that has been reported to support its complete biological signaling.

As disclosed herein analogs of FGF23 having greater potency at the FGF receptor are desirable to enhance the efficacy of FGF23 mediated therapies.

### SUMMARY

In accordance with one embodiment of the present disclosure a method of identifying an optimized FGF23 analog is provided. In one embodiment the method of identifying an optimized FGF23 analog is based on analyzing the C-terminal C26 (SEQ ID NO: 1) and C28 (SEQ ID NO: 2) amino acid peptide fragments of FGF23 (SEQ ID NO: 29) for determining the structure-activity relationship for protein FGF23.

The 28 amino acid FGF23212⁻²³⁹ sequence (C28; SEQ ID NO: 2) was discovered to possess appreciable homology to the reported KL-binding peptide C26 in the extended C-terminus of the protein. C28 was determined to be an independent regulator of FGF23's interaction with the FGFR/KL-complex as first witnessed by inhibition of FGF23 action. Peptide 6 (SEQ ID NO:11) is a refined FGF23 KL-interacting sequence which is enhanced by one order of magnitude in its ability to block in vitro FGF23 activity relative to the native C26 or C28. It also antagonizes endogenous FGF23 action in mice. Unexpectedly, it was found that unlike the KLB-binding peptides, the KL-peptide antagonists maintain their function when shortened to approximately half the length. The natural importance of the second KL-site for FGF23-protein function is exemplified in the FGF23 A¹⁸⁸ analog which preserves bioactivity, despite the loss of the first site C26. When both KL-interaction sites are inactivated, the protein is inactive as determined by in vitro and in vivo studies.

In accordance with one embodiment a peptide exhibiting antagonist activity against FGF23 binding to Klotho is provided. In one embodiment the peptide comprising an amino acid sequence selected from the group consisting of SEQ ID NO: 15, SEQ ID NO: 16, SEQ ID NO: 17 or a peptide that differs from SEQ ID NO: 15, SEQ ID NO: 16, SEQ ID NO: 17 by 1, 2 or 3 amino acid substitutions. In some embodiments the peptide comprises SEQ ID NO: 15. In some embodiments the peptide comprises SEQ ID NO: 15 with one amino acid substitution. In some embodiments the peptide comprises SEQ ID NO: 15 with two amino acid substitutions. In some embodiments the peptide comprises SEQ ID NO: 15 with three amino acid substitutions.

In some embodiments the peptide comprises SEQ ID NO: 16. In some embodiments the peptide comprises SEQ ID NO: 16 with one amino acid substitution. In some embodiments the peptide comprises SEQ ID NO: 16 with two amino acid substitutions. In some embodiments the peptide comprises SEQ ID NO: 16 with three amino acid substitutions. In some embodiments the peptide comprises SEQ ID NO: 17. In some embodiments the peptide comprises SEQ ID NO: 17 with one amino acid substitution. In some embodiments the peptide comprises SEQ ID NO: 17 with two amino acid substitutions. In some embodiments the peptide comprises SEQ ID NO: 17 with three amino acid substitutions. In some embodiments the peptide is a monomer. In some embodiments the peptide is a peptide mixture comprising more than one monomer.

In some embodiments the present invention provides a peptide antagonist of FGF23 KL binding activity wherein the peptide antagonist is 16 amino acids long. In some embodiments the present invention provides a peptide antagonist of FGF23 KL binding activity wherein the peptide antagonist is less than 16 amino acids long.

Optionally the peptide is a dimer formed by covalent linkage between two peptides independently selected from the group consisting of SEQ ID NO: 15, SEQ ID NO: 16, SEQ ID NO: 17 or a peptide that differs from SEQ ID NO: 15, SEQ ID NO: 16, SEQ ID NO: 17 by 1, 2 or 3 amino acid substitutions. In one embodiment the dimer is a heterodimer comprising two different peptides covalently linked to one another. In one embodiment the dimer is a homodimer comprising two identical peptides covalently linked to one another. In one embodiment the two peptides are linked head to tail. In one embodiment the two peptides are linked head to head, or tail to tail. In one embodiment the two peptides are linked to one another via a linker.

In one embodiment dimers are provided comprising any of the two FGF23 C26 or C28 peptides derivatives disclosed herein, linked together via a disulfide bond. More particularly, in one embodiment the peptides of the dimer are modified to comprise a C-terminal extension of 1, 2, 3, 4, 5, 1-10, or 1-5 amino acids where C-terminal extension is terminated with a cysteine, and the dipeptide is formed by a disulfide linkage through the C-terminal cysteine residues of the respective peptides. In one embodiment the dimers are formed by a disulfide bond linking the respective side chains of a C-terminal cysteine of two peptides independently selected from the group consisting of SEQ ID NO: 15, SEQ ID NO: 16, SEQ ID NO: 17 or a peptide that differs from SEQ ID NO: 15, SEQ ID NO: 16, SEQ ID NO: 17 by 1, 2 or 3 amino acid substitutions, wherein each of the peptides of the dimer have been appended with a C-terminal extension that terminates with a cysteine, optionally wherein the C-terminal extension is a pentapeptide C-terminal extension optionally wherein the pentapeptide is GPEGC.

Substituting the native sequence of SEQ ID NO: 1 and/or SEQ ID NO: 2 of FGF23 (SEQ ID NO: 29) with any of the amino acids selected from the group consisting of SEQ ID NO: 15, SEQ ID NO: 16, SEQ ID NO: 17 or a peptide that differs from SEQ ID NO: 15, SEQ ID NO: 16, SEQ ID NO: 17 by 1, 2 or 3 amino acid substitutions is anticipated to produce an FGF analog that has higher potency at the FGF receptor than native FGF23. Accordingly, in one embodiment an agonist analog of FGF23 is provided having enhanced potency at the FGF receptor.

In one embodiment, a patient in need of FGF23 binding KL antagonism is administered a peptide of the present invention. In accordance with one embodiment an improved method for treating bone-mineral diseases is provided. The method comprises the steps of administering to a patient an FGF23-based peptide antagonist as disclosed herein in an amount therapeutically effective for treating bone-mineral diseases. In accordance with one embodiment a method of treating bone mineral diseases is provided wherein the method comprises administering an FGF23-based antagonist as disclosed herein to a patient in need of such therapy.

### BRIEF DESCRIPTION OF THE DRAWINGS

FIGS. 1A-1F Structure activity analysis among the two KL-interacting peptides C26 and C28. FIG. 1A provides a schematic representation of FGF23 showing the Amino terminal FGF23 core (amino acids 25-179) and the C-terminal portion that comprises two Klotho interaction sites “C26” (amino acids 180-205) and “C28” (amino acids 212-239); FIGS. 1B-1F are graphs presenting FGF23-antagonism measured by the relative p-Erk levels in 293/KL cells with increasing doses of the peptide antagonist. (FIG. 1B) compares C26 and C28; FIG. 1C is a graph showing the effect of the substitutions from C28 sequence into the C26 sequence on the peptide activity, comparing peptides C26(●), 1(▪), 2( [symbol] ), 3(▴), and 4(○); FIG. 1D is a graph showing the effect of the substitutions from C28 sequence in the C26 sequence on the peptide activity, C28(●), 5(▪), 6( [symbol] ), and 7(▴); FIGS. 1E & 1F are graphs showing the activity of shortened FGF23 C-terminal peptide antagonist in vitro. For FIG. 1E peptides C26(●), 8(▪), 9( [symbol] ) are compared; For FIG. 1F peptides 6(●), 10(▪), 11( [symbol] ), and 12(▴). The graphs demonstrate a representative curve for normalized p-Erk response with respect to the standard peptide's response, mean ±SD, n=3. Sequence information and potency values presented in Table 1 and 2.

FIG. 2 provides a sequence alignment of the C-terminal sequences of FGF19 (SEQ ID NO: 27, FGF21 (SEQ ID NO: 28), FGF23 180-205 (SEQ ID NO: 1) and FGF23 212-239 (SEQ ID NO: 2).

FIG. 3 is a graph of data demonstrating that mouse kidney gene expression is modulation by an FGF23-antagonist peptide. WT mice were injected with the FGF23 antagonist peptide 6 at the dose of 30 mg/kg body weight, and kidneys were harvested at 3- or 24-hours post-treatment (n=6 per group). The gene expression markers relevant to FGF23-activity were measured by quantitative real time PCR Cyp24a1 (●) and Cyp27b1(▪). The data is presented as a line graph where the mean value with SEM is plotted for each gene. The mice injected with saline (n=6) for 24 h were used as control to establish baseline. One-way ANOVA with Tukey's comparison was employed where statistical significance of *P<0.05 vs saline, and ##P<0.01 vs 3 h treatment was calculated.

FIGS. 4A & 4B provide data relating to mutation of the C-terminal FGF23 peptides to assess their KL-interaction site by study of FGF23-activity antagonism in 293/KL cells. FGF23 C-terminal peptides tested for their ability to block FGF23-signaling measured by relative p-Erk levels are shown in FIG. 4A for peptides C26 (●), C28(▪), C26A1(T [symbol] , FGF23¹⁸⁰⁻²⁰⁵, A¹⁸⁸), C28A2(▴, FGF23²¹²⁻²³⁹, A²²²); and in FIG. 4B for peptides C72(●, FGF23¹⁸⁰⁻²⁵¹), C72A1(▪, FGF23¹⁸⁰⁻²⁵¹, A¹⁸⁸), C72 A2 ( [symbol] , FGF23¹⁸⁰⁻²⁵¹, A²²²), C72A1A2(▴, FGF23¹⁸⁰⁻²⁵¹, A^{188,222}). The graphs demonstrate a representative curve for normalized p-Erk response with respect to the standard peptide's response (FIG. 4A) C26, and (FIG. 4B) C72 respectively, mean ±SD, n=3. Sequence information and potency values presented in Table 1.

FIGS. 5A-5D provide data relating to the activities of FGF23-analogs containing one or two active KL-interaction sites by in vitro and in vivo methods. FIG. 1A is a graph presenting an in vitro assessment of FGF23-signaling activity as measured by relative p-Erk levels in 293/KL cells, FGF23(●), FGF23 A¹⁸⁸ (▪), FGF23 A²²² ( [symbol] ), FGF23 A^{188,222} (▴), the graph demonstrates a representative curve for normalized p-Erk response with reference to the standard FGF23 response, mean ±SD, n=3, the potency values are presented in Table 3. FIGS. 5B-5D are scatter plots presenting an in vivo assessment of FGF23-activity in mice. WT mice were treated with 0.5 mg/kg body weight with either FGF23, FGF23 A¹⁸⁸, FGF23 A²²², FGF23 A^{188,222,} or vehicle, n=5 or 10/group, and post 3-hours the gene expression changes were studied by quantitative real time PCR for target genes, FIG. 5B represents measurement of Cyp24a1, FIG. 5C represents measurement of Cyp27b1, and FIG. 5D represents measurement of Egr1. The data is presented as a scatter plot where each dot represents one animal, mean ±SEM. One-way ANOVA with Tukey's comparison was employed for statistical analyses where***P<0.001 vs. vehicle; #P<0.05, ##P<0.01, ###P<0.001 vs FGF23; and $$$P<0.001 vs FGF23 A^{188,222} was calculated.

FIGS. 6A & 6B provide data relating to the activities of various acylated FGF23-analogs comprising peptide 6 (SEQ ID NO 11) acylated at the N-terminus or C-terminus with a C16 and C18. FIG. 6A, 6(●), 6N-C16 (▪), 6N-C18 ( [symbol] ), or C-terminus FIG. 6B, 6(●), 6C-C16 (▴), 6C-C18(○). FIG. 6C is a table presenting the IC50 values for each tested compound. The structure of the tested acylated peptides are provided in FIGS. 6D-6G, respectively for 6N-C16, 6N-C18, 6C-C16 and 6C-C18.

### DETAILED DESCRIPTION

Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein the term “amino acid” encompasses any molecule containing both amino and carboxyl functional groups, wherein the amino and carboxylate groups are attached to the same carbon (the alpha carbon). The alpha carbon optionally may have one or two further organic substituents. An amino acid can be designated by its three-letter code, one letter code, or in some cases by the name of its side chain. For example, a non-canonical amino acid comprising a cyclohexane group attached to the alpha carbon is termed “cyclohexane” or “cyclohexyl.” For the purposes of the present disclosure designation of an amino acid without specifying its stereochemistry is intended to encompass either the L or D form of the amino acid, or a racemic mixture. However, in the instance where an amino acid is designated by its three letter code (i.e., Lys), such a designation is intended to specify the native L form of the amino acid, whereas the D form will be specified by inclusion of a lower case d before the three letter code or single code (i.e., dLys or dK).

As used herein the term “non-coded (non-canonical) amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

A “bioactive peptide” refers to peptides which can exert a biological effect in vitro and/or in vivo. As used herein a general reference to a peptide is intended to encompass peptides that have modified amino and carboxy termini. For example, an amino acid sequence designating the standard amino acids is intended to encompass standard amino acids at the N- and C- terminus as well as a corresponding hydroxyl acid at the N-terminus and/or a corresponding C-terminal amino acid modified to comprise an amide group in place of the terminal carboxylic acid.

As used herein an “acylated” amino acid is an amino acid comprising an acyl group which is non-native to a naturally occurring amino acid, regardless of the means by which it is produced. Exemplary methods of producing acylated amino acids and acylated peptides are known in the art and include acylating an amino acid before inclusion in the peptide or peptide synthesis followed by chemical acylation of the peptide. In some embodiments, the acyl group causes the peptide to have one or more of (i) a prolonged half-life in circulation, (ii) a delayed onset of action, (iii) an extended duration of action, (iv) an improved resistance to proteases, such as DPP-IV, and (v) increased potency at a receptor for FGF.

As used herein, an “alkylated” amino acid is an amino acid comprising an alkyl group which is non-native to a naturally occurring amino acid, regardless of the means by which it is produced. Exemplary methods of producing alkylated amino acids and alkylated peptides are known in the art and including alkylating an amino acid before inclusion in the peptide or peptide synthesis followed by chemical alkylation of the peptide.

As used herein, the term “prodrug” is defined as any compound that undergoes chemical modification before exhibiting its pharmacological effects.

As used herein a “receptor” is a molecule that recognizes and binds with specific molecules in a high affinity interaction, producing some biological effect (either directly or indirectly) in a cell, or on the cells and/or tissues of the host organism. A “cellular receptor” is a molecule on or within a cell that recognizes and binds with specific molecules, producing some effect (either directly or indirectly) in the cell.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “phosphate buffered saline” or “PBS” refers to aqueous solution comprising sodium chloride and sodium phosphate. Different formulations of PBS are known to those skilled in the art but for purposes of this invention the phrase “standard PBS” refers to a solution having have a final concentration of 137 mM NaCl, 10 mM Phosphate, 2.7 mM KCl, and a pH of 7.2-7.4.

As used herein the term “pharmaceutically acceptable salt” refers to salts of compounds that retain the biological activity of the parent compound, and which are not biologically or otherwise undesirable. Many of the compounds disclosed herein can form acid and/or base salts by virtue of the presence of amino and/or carboxyl groups or groups similar thereto.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as subcutaneous, intramuscular, intraspinal, or intravenous.

As used herein an amino acid “substitution” refers to the replacement of one amino acid residue by a different amino acid residue.

As used herein, the term “conservative amino acid substitution” is defined herein as exchanges within one of the following five groups:
I. Small aliphatic, nonpolar or slightly polar residues: Ala, Ser, Thr, Pro, Gly;
II. Polar, negatively charged residues and their amides: Asp, Asn, Glu, Gln;
III. Polar, positively charged residues: His, Arg, Lys; Ornithine (Orn)
IV. Large, aliphatic, nonpolar residues: Met, Leu, Ile, Val, Cys, Norleucine (Nle), homocysteine (hCys)
V. Large, aromatic residues: Phe, Tyr, Trp, acetyl phenylalanine, napthylalanine (Nal)

As used herein the general term “polyethylene glycol chain” or “PEG chain”, refers to mixtures of condensation polymers of ethylene oxide and water, in a branched or straight chain, represented by the general formula H(OCH₂CH₂)ₖOH, wherein k is at least 2.

As used herein the term “miniPEG” or “OEG” defines a functionalized polyethylene compound comprising the structure:

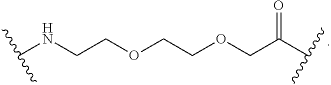 [SMILES: *NCCOCCOCC(*)=O]

As used herein the term “pegylated” and like terms refers to a compound that has been modified from its native state by linking a polyethylene glycol chain to the compound. A “pegylated polypeptide” is a polypeptide that has a PEG chain covalently bound to the polypeptide.

As used herein a “linker” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties, and enzyme-cleavable groups.

As used herein a “dimer” is a complex comprising two subunits covalently bound to one another via a linker. The term dimer, when used absent any qualifying language, encompasses both homodimers and heterodimers. A homodimer comprises two identical subunits, whereas a heterodimer comprises two subunits that differ.

The term “C₁-Cₙalkyl” wherein n can be from 1 through 6, as used herein, represents a branched or linear alkyl group having from one to the specified number of carbon atoms. Typical C₁-C₆ alkyl groups include, but are not limited to, methyl, ethyl, n-propyl, iso-propyl, butyl, iso-butyl, sec-butyl, tert-butyl, pentyl, hexyl and the like.

Physiological conditions as disclosed herein are intended to include a temperature of about 35 to 40° C. and a pH of about 7.0 to about 7.4, and more typically include a pH of 7.2 to 7.4 and a temperature of 36 to 38° C. Since physiological pH and temperature are tightly regulated in humans within a highly defined range, the speed of conversion from dipeptide/drug complex (prodrug) to drug will exhibit high intra and interpatient reproducibility.

As used herein the term “patient” without further designation is intended to encompass any warm blooded vertebrate domesticated animal (including for example, but not limited to livestock, horses, cats, dogs and other pets) and humans.

### REPRESENTATIVE EMBODIMENTS

Beneficial pharmacology observed in preclinical models indicate that FGF23 and its analogs hold promise as innovative therapeutics for treating metabolic disorders. However, analogs of FGF23 having greater potency at the FGF receptor are needed to enhance the efficacy of FGF23 mediated therapies.

At a molecular level, FGF23 interacts with the FGF receptor only in tissues expressing the cofactor Klotho. Accordingly, one approach to enhance the potency of FGF23 analogs at the FGF23 receptor would be to modify FGF23 to enhance its interaction with Klotho.

The amino acid sequence of FGF23 (SEQ ID NO: 29) is as follows:

| | | | | |
|---|---|---|---|---|
| 10 | 20 | 30 | 40 | 50 |
| MLGARLRLWV | CALCSVCSMS | VLRAYPNASP | LLGSSWGGLI | HLYTATARNS |
| 60 | 70 | 80 | 90 | 100 |
| YHLQIHKNGH | VDGAPHQTIY | SALMIRSEDA | GFVVITGVMS | RRYLCMDFRG |
| 110 | 120 | 130 | 140 | 150 |
| NIFGSHYFDP | ENCRFQHQTL | ENGYDVYHSP | QYHFLVSLGR | AKRAFLPGMN |
| 160 | 170 | 180 | 190 | 200 |
| PPPYSQFLSR | RNEIPLIHFN | TPIPRRHTRS | AEDDSERDPL | NVLKPRARMT |
| 210 | 220 | 230 | 240 | 250 |
| PAPASCSQEL | PSAEDNSPMA | SDPLGVVRGG | RVNTHAGGTG | PEGCRPFAKFI |

The N-terminal FGF23 core (SEQ ID NO: 30):

| | | | | |
|---|---|---|---|---|
| 10 | 20 | 30 | 40 | 50 |
| | | YPNASP | LLGSSWGGLI | HLYTATARNS |
| 60 | 70 | 80 | 90 | 100 |
| YHLQIHKNGH | VDGAPHQTIY | SALMIRSEDA | GFVVITGVMS | RRYLCMDFRG |
| 110 | 120 | 130 | 140 | 150 |
| NIFGSHYFDP | ENCRFQHQTL | ENGYDVYHSP | QYHFLVSLGR | AKRAFLPGMN |
| 160 | 170 | 180 | | |
| PPPYSQFLSR | RNEIPLIHFN | TPIPRRHTR | | |

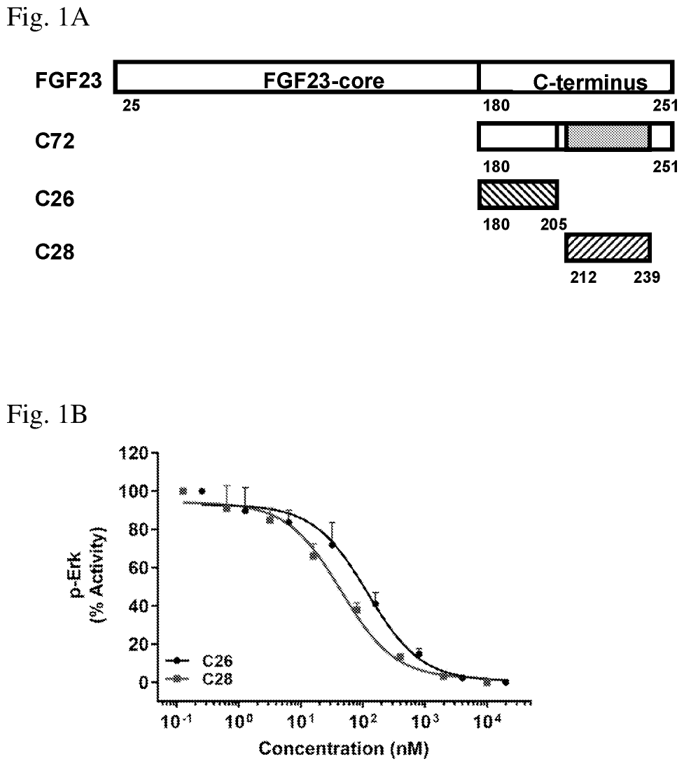

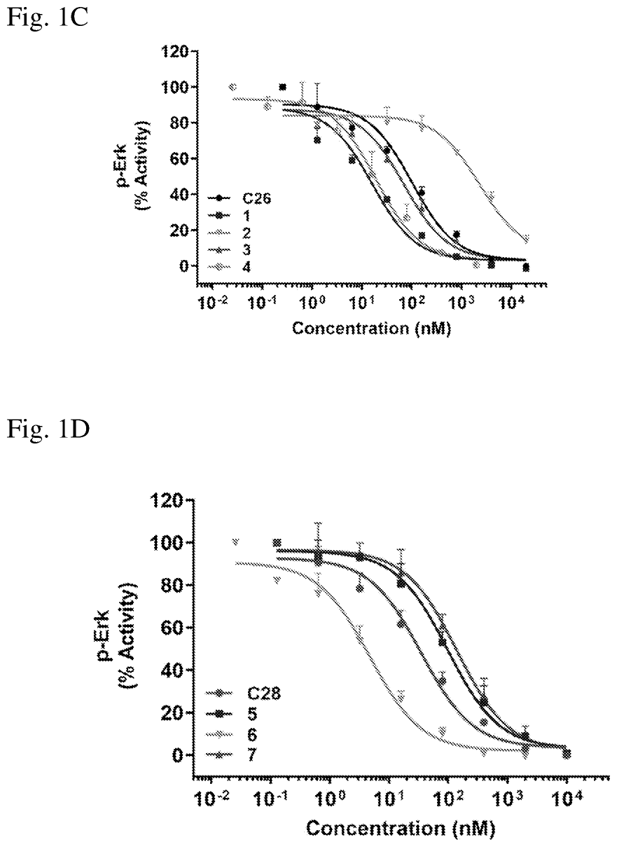

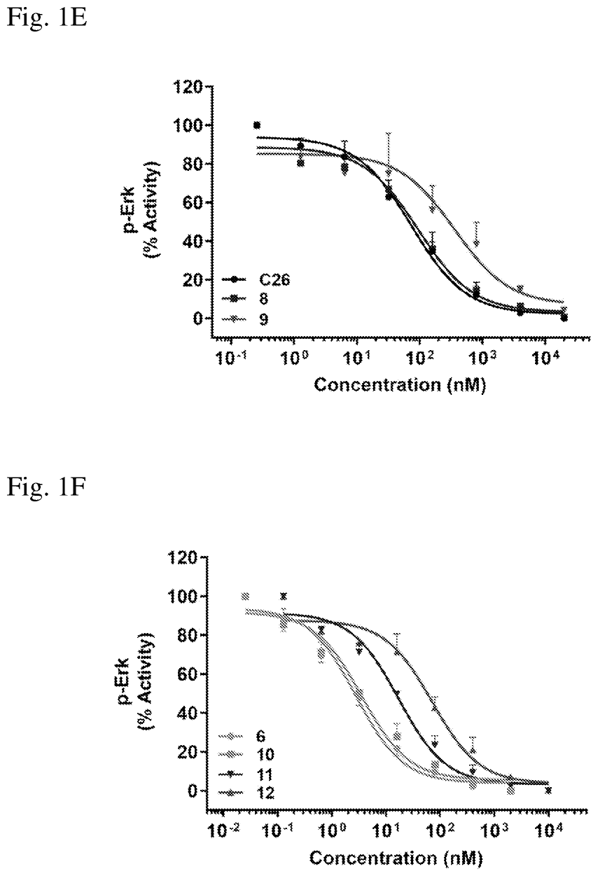

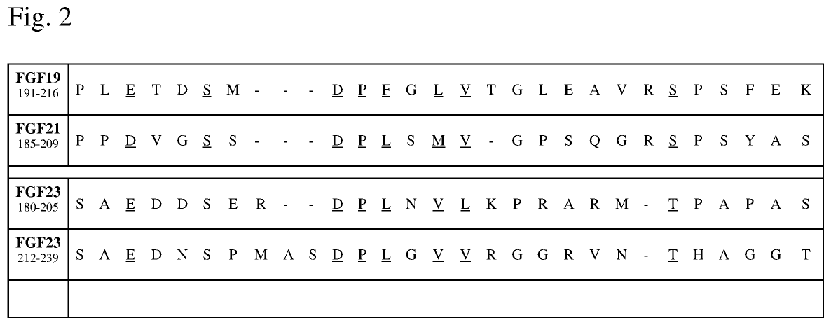

N-terminal FGF23 core, comprising residues 25-179 of FGF23 interacts with the FGF receptor. The C-terminus of FGF23 is believed to play a key role in binding with Klotho (KL). A C-terminal peptide (C26; SEQ ID NO: 1) was known to interact with KL, however, based upon sequence homology, an additional novel C-terminal FGF23²¹²⁻²³⁹ peptide sequence (C28, also referred to as second KL-site; SEQ ID NO: 2) was identified as a potential KL interaction site (FIG. 1A). Sequence alignment of C28 with the well-characterized KL-binding peptide FGF23¹⁸⁰⁻²⁰⁵ (C26, also referred to as first KL-site) displayed 40% identity between the two peptides (Table 1, FIG. 2). The segment (DPLNVLK; SEQ ID NO: 31) that is critical for the interaction of C26 and KL was highly conserved in C28 (DPLGVVR; SEQ ID NO: 32, three non-conserved residues in bold font). Four of the seven amino acids were identical and the last two residues homologous, with only a centrally located glycine as a notable exception (Table 1).

TABLE 1

| Name/ | | | IC₅₀ ± SD |
|---|---|---|---|
| SEQ ID No | Position | Sequence | (nM) |
| C26 | FGF23¹⁸⁰⁻²⁰⁵ | SAEDD SER-- DPLNV LKPRA RMTPA | 92.3 ± 27.9 |
| SEQ ID NO: 1 | | PAS | |
| C28 | FGF23²¹²⁻²³⁹ | SAEDN SPMAS DPLGV VRGGR VNTHA | 36.2 ± 12.1 |
| SEQ ID NO: 2 | | GGT | |
| C26 A1 | FGF23¹⁸⁰⁻²⁰⁵, | SAEDD SER-- APLNV LKPRA RMTPA | NC |
| SEQ ID NO: | A¹⁸⁸ | PAS | |
| 22 | | | |
| C28 A2 | FGF23²¹²⁻²³⁹, | SAEDN SPMAS APLGV VRGGR VNTHA | NC |
| SEQ ID NO: | A²²² | GGT | |
| 23 | | | |
| C72 | FGF23¹⁸⁰⁻²⁵¹ | SAEDD SER-- DPLNV LKPRA RMTPA | 1.2 ± 0.5 |
| SEQ ID NO: 3 | | PAS CSQELP SAEDN SPMAS DPLGV | |
| | | VRGGR VNTHA GGT GPEGC RPFAKFI | |
| C41 | FGF23 ¹⁶⁵⁻²⁰⁵ | PLIHFNTPIPRRHTR SAEDD SER-- | 27.8 ± 5.6 |
| SEQ ID NO: 4 | | DPLNV LKPRA RMTPA PAS | |
| C40; | FGF23²¹²⁻²⁵¹ | SAEDN SPMAS DPLGV VRGGR VNTHA | 23.3 ± 7.4 |
| SEQ ID NO: 5 | | GGT GPEGC RPFAKFI | |
| C72 A1 | FGF23¹⁸⁰⁻²⁵¹, | SAEDD SER-- APLNV LKPRA RMTPA | 24.5 ± 11.5 |
| SEQ ID NO: | A¹⁸⁸ | PAS CSQELP SAEDN SPMAS DPLGV | |
| 24 | | VRGGR VNTHA GGT GPEGC RPFAKFI | |
| C72 A2 | FGF23¹⁸⁰⁻²⁵¹, | SAEDD SER-- DPLNV LKPRA RMTPA | |
| SEQ ID NO: | A²²² | PAS CSQELP SAEDN SPMAS APLGV | 33.1 ± 15.8 |
| 25 | | VRGGR VNTHA GGT GPEGC RPFAKFI | |
| C72 A1A2 | FGF23¹⁸⁰⁻²⁵¹ | SAEDD SER-- APLNV LKPRA RMTPA | NC |
| SEQ ID NO: | A^{188,} ²²² | PAS CSQELP SAEDN SPMAS APLGV | |
| 26 | | VRGGR VNTHA GGT GPEGC RPFAKFI | |
| 21C25 | FGF21¹⁸⁵⁻²⁰⁹ | | 75.2 ± 19.0 |

When tested in 293T HEK cells that overexpress the human KL receptor (293/KL), C28 competitively blocked FGF23-signaling at nearly three-fold enhanced potency relative to C26 (FIG. 1B, Table 1). Collectively these results indicate that a novel FGF23-derived peptide (C28) independent of the reported KL-binding site (C26) is equally capable of antagonizing FGF23 in vitro activity. The specificity in KL-dependent signaling was demonstrated by the inability of these FGF23 derived peptides to block FGF21-signaling in 293/KLB cells as compared to FGF21 associated antagonist (FGF21¹⁸⁵⁻²⁰⁹, C25). The sequence homology in these endocrine FGF peptides (FGF23¹⁸⁰⁻²⁰⁵, FGF23²¹²⁻²³⁹, FGF19¹⁹¹⁻²¹⁶ and FGF21¹⁸⁵⁻²⁰⁹) is provided in FIG. 2.

The relative basis for activity in the C26 and C28 peptides was explored to determine whether they possess a common structural core for KL-binding, and if their inhibitory potency could be enhanced. Reciprocal substitutions within the two sequences were made that focused on three subsequences with the greatest variation. The first set of peptides sequentially introduced four subsequences from C28 to C26 (peptides 1-4) as shown in Table 2.

TABLE 2

| Name/SEQ | | | |
|---|---|---|---|
| ID NO: | Position | Sequence | IC₅₀ ± SD (nM) |
| 1/SEQ ID | | SAEDD SPMAS DPLNV LKPRA | 18.7 ± 4.7 |
| NO: 6 | | RMTPA PAS | |
| 2/SEQ ID | | SAEDD SER DPLNV LKGGR VNTPA | 3587.0 ± 1782.7 |
| NO: 7 | | PAS | |
| 3/SEQ ID | | SAEDD SER DPLNV LKPRA RMTHA | 77.7 ± 34.6 |
| NO: 8 | | GGT | |
| 4/SEQ ID | | SAEDD SERAS DPLNV LKPRA | 21.3 ± 11.7 |
| NO: 9 | | RMTPA PAS | |
| 5/SEQ ID | | SAEDN SER DPLGV VRGGR VNTHA | 143.0 ± 77.0 |
| NO: 10 | | GGT | |
| 6/SEQ ID | | SAEDN SPMAS DPLGV VRPRA | 3.7 ± 1.2 |
| NO: 11 | | RMTHA GGT | |
| 7/SEQ ID | | SAEDN SPMAS DPLGV VRGGR | 157.3 ± 47.9 |
| NO: 12 | | VNTPA PAS | |
| 8/SEQ ID | FGF23¹⁸⁶⁻¹⁹⁹ | ERDPLNV LKPRA RM | 97.0 ± 30.3 |
| NO: 13 | | | |
| 9/SEQ ID | FGF23¹⁸⁸⁻¹⁹⁹ | DPLNV LKPRA RM | 542.3 ± 282.0 |
| NO: 14 | | | |
| 10/SEQ ID | | PMAS DPLGV VRPRA RM | 3.8 ± 0.9 |
| NO: 15 | | | |
| 11/SEQ ID | | AS DPLGV VRPRA RM | 17.1 ± 0.6 |
| NO: 16 | | | |
| 12/SEQ ID | | DPLGV VRPRA RM | 70.9 ± 29.5 |
| NO: 17 | | | |
| 13/SEQ ID | C26- | SAEDD SER DPLNV LKPRA RMTPA | 107.3 ± 16.6 |
| NO: 18 | GPEGC | PAS GPEGC | |
| 14/SEQ ID | C28- | SAEDN SPMAS DPLGV VRGGR | 57.5 ± 16.3 |
| NO: 19 | GPEGC | VNTHA GGT GPEGC | |
| 15/SEQ ID | 6-GPEGC | SAEDN SPMAS DPLGV VRPRA | 5.8 ± 1.1 |
| NO: 20 | | RMTHA GGT GPEGC | |
| 16/SEQ ID | 15 A 2.2.2 | SAEDN SPMAS APLGV VRPRA | NC |
| NO: 21 | | RMTHA GGT GPEGC | |
| 17/ | | 13-13 disulfide dimer | 2.2 ± 1.2 |
| 18/ | | 14-14 disulfide dimer | 3.1 ± 1.8 |
| 19/ | | 13-14 disulfide dimer | 2.7 ± 1.3 |
| C26 + C28/ | | Non-covalent mixture | 15.8 ± 2.2 |
| 20/ | | 15-15 disulfide dimer | 1.0 ± 0.4 |
| 21/ | | 16-16 disulfide dimer | NC |
| 22/ | | 15-16 disulfide dimer | 3.4 ± 0.1 |

The substitution with the PMAS²¹⁸⁻²²¹ subs ection of C28 in peptide 1 enhanced the antagonistic potency of C26 by five-fold (FIG. 1C, Table 2). The second substitution of GGRVN²²⁹⁻²³³ residues (peptide 2) decreased the potency of C26 by nearly forty-fold, while the third substitution of HAGGT²³⁵⁻²³⁹ (peptide 3) proved to be of little difference (FIG. 1C, Table 2). The last analog of the set consisted of an insertion of two amino acids AS²²⁰⁻²²¹ (peptide 4) and this produced a four-fold increase in antagonism (FIG. 1C, Table 2). A second set of reciprocal substitutions employing subsections of C26 was introduced to C28 (Table 2). The introduction of ER¹⁸⁶⁻¹⁸⁷ (peptide 5) or PAPAS²⁰¹⁻²⁰⁵ (peptide 7) resulted in a loss of potency by four and six-fold relative to C28, respectively (FIG. 1D, Table 2). In contrast, the substitution with the pentapeptide PRARM¹⁹⁵⁻¹⁹⁹ in peptide 6 provided a nearly ten-fold enhancement to the native antagonism of C28 (FIG. 1D, Table 2). Peptide 6 proved the most potent of this 28-residue set, and was more than twenty-fold enhanced relative to the previously recognized C26 antagonist (Table 1, 2).

A shortened C26 peptide sequence surprisingly found retention of significant activity at a length of fourteen amino acids (peptide 8, FGF23¹⁸⁶⁻¹⁹⁹) and even the twelve amino acid peptide (peptide 9, FGF23¹⁸⁸⁻¹⁹⁹) was a full antagonist of FGF23 but of much reduced potency (FIG. 1E, Table 2). An analogous shortening of the most potent chimeric antagonist peptide 6 revealed similar behavior but with enhanced potency relative to C26 at every peptide length studied (peptides 10, 11, and 12; Table 2). The potency of the shortest optimized peptide composed of twelve amino acids (peptide 12) was comparable to C26 (FIG. 1F, Table 1, 2).

In accordance with one embodiment a peptide exhibiting antagonist activity against FGF23 binding to Klotho is provided wherein said peptide comprises an amino acid sequence selected from the group consisting of SEQ ID NO: 15, SEQ ID NO: 16, SEQ ID NO: 17 and a peptide that differs from SEQ ID NO: 15, SEQ ID NO: 16, or SEQ ID NO: 17 by one or two or three amino acid substitutions. In some embodiments the peptide comprises SEQ ID NO: 15. In some embodiments the peptide comprises SEQ ID NO: 15 with one amino acid substitution. In some embodiments the peptide comprises SEQ ID NO: 15 with two amino acid substitutions. In some embodiments the peptide comprises SEQ ID NO: 15 with three amino acid substitutions.

In some embodiments the peptide antagonist comprises SEQ ID NO: 16. In some embodiments the peptide comprises SEQ ID NO: 16 with one amino acid substitution. In some embodiments the peptide comprises SEQ ID NO: 16 with two amino acid substitutions. In some embodiments the peptide comprises SEQ ID NO: 16 with three amino acid substitutions. In some embodiments the peptide comprises SEQ ID NO: 17. In some embodiments the peptide comprises SEQ ID NO: 17 with one amino acid substitution. In some embodiments the peptide comprises SEQ ID NO: 17 with two amino acid substitutions. In some embodiments the peptide comprises SEQ ID NO: 17 with three amino acid substitutions. In some embodiments the peptide is a monomer. In some embodiments the peptide is a peptide mixture comprising more than one monomer.

In some embodiments the present invention provides a peptide antagonist of FGF23 KL binding activity wherein the peptide antagonist is 16 amino acids long. In some embodiments the present invention provides a peptide antagonist of FGF23 KL binding activity wherein the peptide antagonist is less than 16 amino acids long.

In accordance with embodiment 1 a peptide exhibiting antagonist activity against FGF23 binding to Klotho is provided wherein said peptide comprises a dimer comprising two peptides, wherein each peptide comprises an amino acid sequence independently selected from the group consisting of SEQ ID NO: 11, SEQ ID NO: 15, SEQ ID NO: 16, SEQ ID NO: 17 and a peptide that differs from SEQ ID NO: 11, SEQ ID NO: 15, SEQ ID NO: 16, or SEQ ID NO: 17 by one or two amino acid substitutions, wherein said peptides are covalently linked via a linker, optionally wherein each peptide comprises an amino acid sequence independently selected from the group consisting of SEQ ID NO: 15, and a peptide that differs from SEQ ID NO: 15 by one or two amino acid substitutions.

In accordance with embodiment 2 a dimer in accordance with embodiment 1 is provided wherein the linker is a bond, an amino acid, or peptide and the dimer is in the form of a linear contiguous amino acid sequence.

In accordance with embodiment 3 a dimer in accordance with embodiment 1 is provided wherein the dimer is formed via a disulfide linkage between the side chain of an amino acid of each peptide, optionally wherein each of the two peptides comprising the dimer further comprise a C-terminal extension of 1 to 5 amino acids optionally wherein the C-terminal amino acid of the C-terminal extensions comprise a cysteine and a disulfide bond is formed between the side chains of said cysteine residues.

In accordance with embodiment 4 a dimer in accordance with embodiments 2 or 3 is provided wherein the dimer is a homodimer.

In accordance with embodiment 5 a dimer in accordance with embodiments 2 or 3 is provided wherein the dimer is a heterodimer.

In accordance with embodiment 6 a peptide or dimer in accordance with any one of embodiments 1-5 is provided wherein the peptide or dimer is modified by covalent linkage of one or more C14-C20 alkyl or acyl groups, optionally wherein the acyl group is a C16 or C18 fatty acid or fatty diacid linked to the N-terminus or the C-terminus of the peptide or dimer, optionally via a linker.

In accordance with embodiment 7 a peptide or dimer in accordance with any one of embodiments 1-6 is provided wherein the peptide or dimer is fused to the carboxy terminus of the FGF23 core sequence, optionally wherein the FGF23 core sequence comprises a peptide of SEQ ID NO: 30 or a peptide that differs from SEQ ID NO: 30 by 1 to 3 amino acid substitutions

In accordance with one embodiment a pharmaceutical composition is provided comprising any of the peptides or dimers disclosed herein that exhibit antagonist activity against FGF23 binding to Klotho, preferably at a purity level of at least 90%, 91%, 92%, 93%, 94%, 95%, 96%, 97%, 98% or 99%, and a pharmaceutically acceptable diluent, carrier or excipient. Such compositions may contain a peptide or dimer as disclosed herein at a concentration of at least 0.1-10 mg/ml, or higher. In one embodiment the pharmaceutical compositions comprise aqueous solutions that are sterilized and optionally stored within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

In accordance with embodiment 8 a method for reducing excessive actions of FGF23 is provided wherein said method comprises administering to a patient in need thereof a pharmaceutical composition comprising a peptide of any one of embodiments 1-7 in an amount effective to increase serum phosphate levels.

In accordance with embodiment 9 a method for treating a bone-mineral disease is provided wherein the method comprises administering to a patient in need thereof a pharmaceutical composition comprising a peptide of any one of embodiments 1-7 in an amount effective to treat said disease, optionally wherein the disease is selected from the group consisting of hypophosphatemic rickets/osteomalacia including X-linked hypophosphatemic rickets (XLH) and tumor-induced osteomalacia.

A non-exclusive list of other diseases, disorders, or conditions which may be treated, diagnosed, ameliorated, or prevented with the FGF23 antagonist peptides include: dermal wounds, epidermolysis bullosa, male pattern alopecia, gastric ulcer, duodenal ulcer, erosive gastritis, esophagitis, esophageal reflux disease, inflammatory bowel disease, radiation- or chemotherapy-induced gut toxicity, hyaline membrane disease, necrosis of the respiratory epithelium, emphysema, pulmonary inflammation, pulmonary fibrosis, hepatic cirrhosis, fulminant liver failure, and viral hepatitis. Research has shown several clinically distinct disorders caused by a gain of FGF-23 function leading to hypophosphatemia and the resulting conditions, osteomalacia (defective bone mineralization) or rickets (defective cartilage mineralization). Autosomal dominant hypophosphatemic rickets (ADHR) is caused by mutation of one of two closely spaced arginine residues of FGF-23 (R176 and R179). These residues are near a protease processing site (R179/S180) that is cleaved by the membrane-associated metalloprotease, PHEX, leading to inactivation of FGF-23. Mutant forms of the protein can be resistant to proteolytic cleavage, resulting in overly active FGF23 signaling and FGF23 peptides of the present invention can be used to modulate this condition.

In accordance with one embodiment any of the peptides or dimers disclosed herein that exhibit antagonist activity against FGF23 binding to Klotho can be further modified to have an improved therapeutic index and an in vivo extended time of action when administered to a warm blooded mammal including, for example, homo sapiens. More particularly, in one embodiment the peptides and dimers disclosed herein are modified by the covalent linkage of a fatty acid or fatty diacid of sufficient size to bind serum albumin with high affinity, optionally wherein the fatty acid or fatty diacid a C16-C18 fatty acid or C16-C18 fatty diacid. In accordance with one embodiment one or more lysine resides of the peptide or dimer disclosed herein is modified by the covalent linkage of a C16-C18 fatty acid or C16-C18 fatty diacid the side chain of the lysine. In one embodiment the acylated lysine residue is a lysine that has been added to the amino or carboxy terminus of a peptide or dimer of the present disclosure. In accordance with one embodiment the peptides and dimers disclosed herein are further modified by acylation, wherein the acyl group is linked to the side chain of an amino acid, optionally lysine or cysteine, located at the N-terminus and/or at the C-terminus of the peptide or dimer. In one embodiment the acyl group is of sufficient size to bind serum albumin with high affinity. In one embodiment the acyl group is a C16-C18 fatty acid or C16-C18 fatty diacid, optionally wherein the acyl group is linked via a spacer, including for example a miniPEG spacer.

In accordance with one embodiment the peptides and dimers disclosed herein can be further modified by linkage to a self-cleaving dipeptide wherein an amino acid of the dipeptide is optionally acylated with a fatty-acyl group of sufficient size to bind serum albumin with high affinity.

In a further embodiment amino acid “A” of the self-cleaving dipeptide “A-B” is a lysine residue acylated with a C16-C30 fatty acid or C16-C30 diacid. In one embodiment A and B are selected to provide a chemical cleavage half-life (t1/2) of A-B from the peptides or dimers disclosed herein of at least about 24 hours to about 240 hours, about 48 hours to about 168 hours, or about 48 to about 120 hours, or about 70 to about 100 hours in standard PBS solution under physiological conditions. In one embodiment the C-terminal amino acid of any of the peptides disclosed herein can be modified to replace the native carboxyl group with an amide.

In one embodiment the peptides and dimers disclosed herein are covalently linked to a self-cleaving dipeptide of the structure:

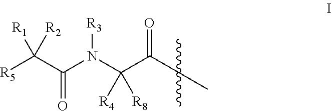

wherein
R₁, is a side chain selected from the group consisting of C₁-C₁₈alkyl, (C₁-C₄alkyl)OH, (C₁-C₄alkyl)SH, (C₁-C₄alkyl)COOH, and (C₁-C₄alkyl)NH₂, optionally wherein a C16-C20 fatty acid or a C16-C20 diacid is covalently linked to said side chain;
R₂, R₄ and Rs are independently H, or C₁-C₄alkyl;
R₃ is C₁-C₄ alkyl, or R₄ and R₃ together with the atoms to which they are attached form a 5 or 6 member heterocyclic ring; and
R5 is NH₂, with the proviso that when R₄ and R₃ together with the atoms to which they are attached form a 5 or 6 member heterocyclic ring, R₂ is not H. In one embodiment the acylated amino acid of A is independently selected from an amino acid having the general structure of

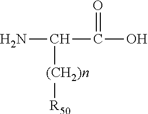

wherein n is an integer selected from the range of 1-4 and R₅₀ is selected from the group consisting of NH—CO(CH₂)₁₄₋₂₀COOH, NH-[spacer]-CO(CH₂)₁₄₋₂₀COOH, S(CH₂)₁₄₋₂₀COOH and S-[spacer]-CO(CH₂)₁₄₋₂₀COOH. In one embodiment the acylated amino acid of A is independently selected from lysine, d-lysine, ornithine, cysteine or homocysteine wherein the side chain of said acylated amino acid is covalently linked to a C16-C22 fatty acid or C16-C22 diacid optionally through a spacer comprising an amino acid or dipeptide. In one embodiment the spacer comprises a gamma glutamic acid. In one embodiment the optional spacer comprises two gamma glutamic acids, optionally wherein the two gamma glutamic acids are joined to one another via an intervening functionalized PEG polymer, [COCH₂(OCH₂CH₂)ₖHN]_{q}, wherein k and q are each integers independently selected from 1, 2, 3, 4, 5, 6, 7 or 8. In a further embodiment the self-cleaving dipeptide has the structure of formula I wherein R₁, is (C₁-C₄ alkyl)NH—CO(CH₂)₁₄₋₂₀COOH or (C₁-C₄ alkyl)NH-[spacer]-CO(CH₂)₁₄₋₂₀COOH; R₂ and R₈ are each H; R₄ is H, or CH₃; R₃ is CH₃ and R₅ is NH₂, optionally wherein the first amino acid of the self-cleaving dipeptide is an amino acid in the D-stereochemical configuration and the spacer is selected from the group consisting of a gamma glutamic acid, a gamma glutamic acid-gamma glutamic acid dipeptide, and COCH₂(OCH₂CH₂)₂HN.

### EXAMPLES

The optimized FGF23 C-terminal peptide is a competitive in vivo FGF23-antagonist

C72 and C26 have been reported to modestly modulate serum phosphate levels in normal rodents. In the parallel KLB-dependent system, FGF19 C-terminal peptides can competitively block FGF19 or FGF21 protein activity at their target organs such as pancreas and adipose tissue. These studies establish a precedent that Klotho co-receptor associated in vivo bioactivity can be modified by employing peptides that function as FGF-antagonists in vitro.

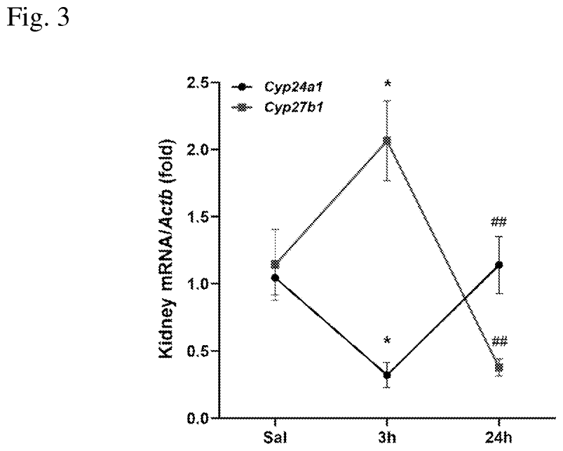

FGF23 lowers circulating 1, 25-dihydroxy vitamin D via the renal activation of vitamin D 24-hydroxylase (Cyp24a1), and simultaneous suppression of vitamin D 1α-hydroxylase (Cyp27b1). To determine whether the FGF23 C-terminal peptide can reverse these effects by blocking endogenous FGF23 action, wild type mice were treated with peptide 6 via a single i.p. injection. After 3 hours, the kidneys were harvested to quantitate gene expression of the vitamin D regulating enzymes. The Cyp24a1 mRNA level was significantly reduced while that of Cyp27b1 was reciprocally increased, as compared to the saline controls (P<0.05). The same measurements at the 24-hour post dosing time point revealed the expression levels of these target genes having returned to baseline values, and significantly different from the 3-hour group, P<0.01; FIG. 3).

Site directed mutagenesis to validate the interaction of the second FGF23 KL-site

Structural and biochemical studies have shown that mutation of a single Asp188 in the C26 peptide region can inactivate the FGF23 protein. In an analogous system, FGF19 and FGF21 proteins also utilize the corresponding sequence aligned Asp192 to interact with KLB, and substitution to alanine renders the corresponding C-terminal peptides non-functional as KLB-dependent antagonists. Therefore, an analogous approach was used to study the interaction between the C-terminus of FGF23 and KL.

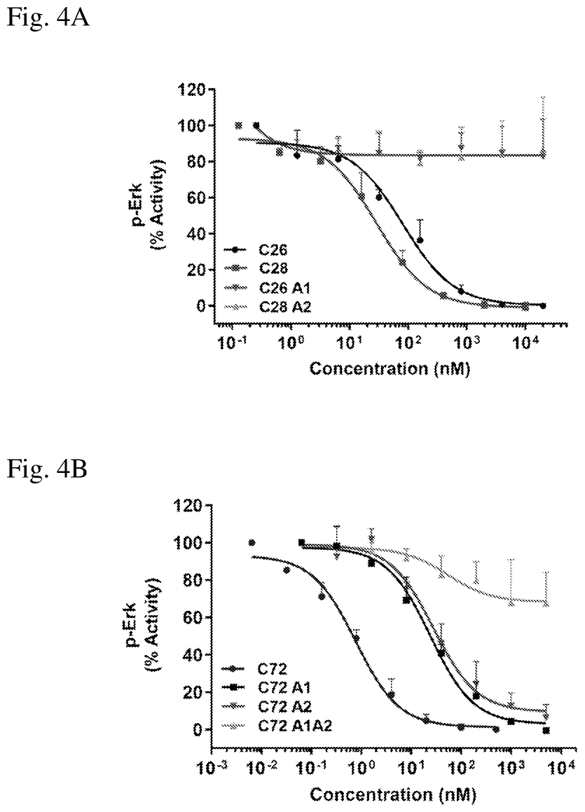

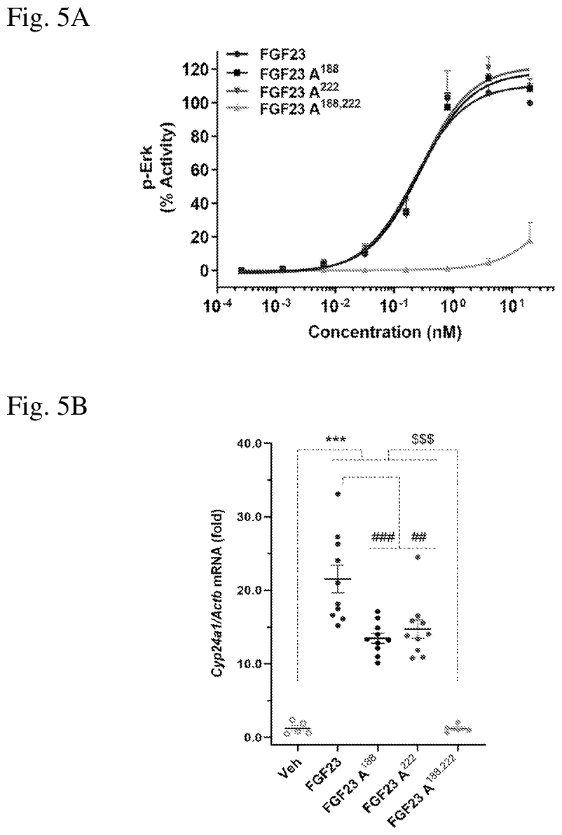

To test whether C28 utilizes a comparable binding epitope as C26 for interacting with FGFR/KL-complex, the central aspartic acid residues (Asp188 or Asp222) were site-specifically substituted with an alanine (C26 A1 or C28 A2, Table 1). There was a dramatic loss of antagonistic activity in each peptide resulting from the aspartic acid replacement with alanine (FIG. 4A, Table 1). Similarly this approach was applied to the fuller length FGF23 C-terminal peptide of 72-aa length (C72), which harbors both the C26 and C28 peptide sites. The C72 peptide potency proved seventy and thirty-fold higher than the individual C26 and C28 peptides respectively (Table 1). To confirm that the increased potency of C72 was indeed derived from the presence of two functional KL-sites (C26 and C28) and not the extended peptide length, two additional peptides were studied. The peptide with an N-terminal extension of fifteen residues to C26 was termed C41, and another peptide C40 comprised a C-terminal extension of twelve residues to C28 (Table 1). Each of these peptides revealed a subtly higher potency when compared to the native C26 or C28, respectively (two to three-fold, Table 1). However, neither of the extended peptides represents a potency comparable to the native C72 with an IC50 of 1.2 nM. Site specific inactivation of each site in C72 by singularly substituting Asp188 or Asp222 with alanine (C72 A1 or C72 A2) was performed. In each instance, there was a reduction in inhibitory potency of FGF23 by more than twenty-fold, but each peptide remained of equal or greater potency than C26 or C28 (FIG. 4B, Table 1). When a double substitution of Asp188 and Asp222 to alanine was made (C72 A1A2), a complete loss of activity was observed (FIG. 4B, Table 1).

To further interrogate whether the superior potency of C72 derives from two KL-interacting sites or alternatively is a function of increased native sequence size, a series of dimeric peptide analogs of non-native structure were engineered. The peptides C26, C28 or 6 were appended with a pentapeptide C-terminal extension that terminated with a cysteine (Table 2) yielding peptides 13, 14 and 15 respectively. The extension represents a spacer derived from the native FGF23 sequence (GPEGC240-244), that follows the C28 sequence. The disulfide homodimers of peptides 13 and 14 were prepared by the direct oxidation of the terminal cysteine to produce peptides 17 and 18 (Table 2). The FGF23-antagonistic potencies of these dimeric peptides were much enhanced (twenty- to fifty-fold) compared to the corresponding monomers and nearly as potent as C72 (Table 1, 2). Peptide 19 is the heterodimer formed by 13 and 14, possessing the two native KL-interaction sites of C72, and was indistinguishable from the activity of the two homodimers (17 and 18) (Table 2). When the most potent single site FGF23-antagonist (peptide 15) was homodimerized to generate peptide 20, it was of no greater potency than C72, implying that the native C72 represents an upper limit of FGF23-antagonistic potency (Table 1, 2).

A series of negative controls performed to expectations where the peptides 13, 14 and 15 proved similar to the parent peptides without the pentapeptide extension (Table 1, 2). Peptide 16 is identical to 15 except it possesses the deactivating Asp222Ala substitution, and was found to be inactive as expected when tested as a monomer or a dimer (21) (Table 2). Additionally, peptide 22 which constitutes a heterodimer of the highly potent peptide 15 and the inactive peptide 16 was reduced in potency relative to C72 and peptide 20 (Table 1, 2). These results demonstrate that two KL-binding sites increase KL-antagonism potency but that there is an upper limit to what is achievable. The magnitude in gain of potency when transitioning from a monomeric to dimeric entity is prominent in the cases where dimers are formed from non-optimal monomers (17, 18 and 19), and diminishes when an optimized monomer is dimerized (20). Finally, the non-covalent mixing of C26 and C28 demonstrates a slight potency enhancement, however the magnitude of the change is much smaller than what is observed through a covalent linkage (Table 2).

Demonstration of FGF23 agonism as mediated by the second and novel KL-site

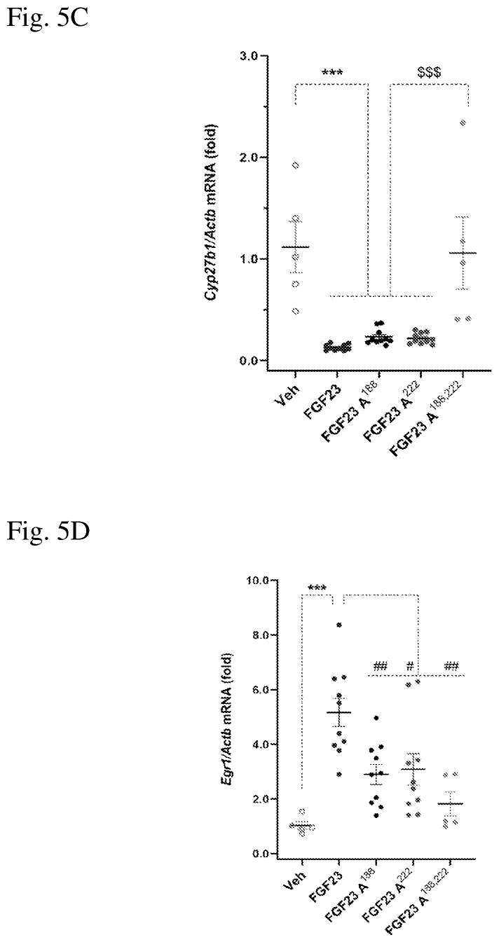

Whether FGF23-based agonism can employ each of the two KL-interaction sites as witnessed in the C72 peptide antagonist was studied. To do so, a selective mutation at position Asp188 or Asp222 was made in the native FGF23. The protein analogs were tested for stimulation of Erk-phosphorylation in 293/KL cells and the A222 mutant proved of comparable potency to native protein. This is consistent with prior reports where C-terminal sequence deletion after residue 205 position had no apparent effect on cellular signaling (FIG. 5A, Table 3). Surprisingly, the FGF23, A188 analog also retained full potency with no discernible difference relative to native FGF23. Asp188 residue is purported to be singularly essential for KL-binding and FGF23 activity, and its importance was substantiated by the lack of antagonism when the C26 peptide was similarly mutated from Asp to Ala (FIG. 4A). This result suggests that in the absence of a functional C26 KL-site, the downstream C28 KL-site enables FGF23 to productively signal through the FGPR/KL complex. When the Asp188 and Asp222 residues of FGF23 were both mutated to alanine (A188,222 analog), the bioactivity was abrogated (FIG. 5A, Table 3). To confirm that these observations were not resulting from unexpected changes in higher order structure that was not apparent in biosynthesis, purification, or formulation of the mutant proteins their biophysical integrity were directly assessed by thermal denaturation.

TABLE 3

| | | EC₅₀ ± SD |
|---|---|---|
| Name | Description | (nM) |
| FGF23 | FGF23²⁵⁻²⁴⁰ | 0.16 ± 0.05 |
| FGF23 A¹⁸⁸ | FGF23²⁵⁻²⁴⁰, A¹⁸⁸ | 0.28 ± 0.08 |
| FGF23 A²²² | FGF23²⁵⁻²⁴⁰, A²²² | 0.29 ± 0.12 |
| FGF23 A^{188,222} | FGF23²⁵⁻²⁴⁰, A^{188,222} | NC |
| FGF21 | FGF21²⁹⁻²⁰⁹ | NC |
| FGF21-23 C72 | FGF21²⁹⁻¹⁸⁴ FGF23¹⁸⁰⁻²⁵¹ | 0.20 ± 0.04 |
| FGF21-23 C26 | FGF21²⁹⁻¹⁸⁴ FGF23¹⁸⁰⁻²⁰⁵ | 197.3 ± 166.5 |

To further explore the function of the second KL-site, the prospect was investigated that possessing two sites might prove advantageous in a specific context where the FGFR binding element is less than optimal. The N-terminal domain of the endocrine FGFs primarily determines the FGFR-activation while the C-terminal sequence guides the co-receptor specificity with a Klotho co-receptor, KL or KLB. The receptor complex specificity among the endocrine FGF-proteins can be modulated by altering their C-terminal domains. Consequently, two FGF21-23 chimeric protein analogs were prepared containing the FGF21 N-terminal core with its C-terminal KLB-binding peptide replaced with an FGF23 C-terminal peptide sequence constituted by C72 (FGF21-23 C72) or C26 (FGF21-23 C26) (Table 3). Native FGF21 was unable to activate the FGFR/KL complex as expected but the FGF21-23 C72 analog demonstrated a sub-nanomolar FGFR-activation (EC50 0.2 nM) and partial agonism as compared to native FGF23 (Table 3). The single KL-site FGF21 analog (FGF21-23 C26, EC50 197.3 nM) was a thousand-fold less potent than the double KL-site FGF21-23 C72 analog (Table 3). These results confirm that while the individual KL-site mutations in FGF23 did not diminish in vitro efficacy, the absence of the second site in an unoptimized FGFR-ligand dramatically decreases agonism.

To test whether C28 as the second KL-interaction site can promote FGF23 in vivo activity, the single site FGF23-analogs were assessed for their ability to alter kidney gene expression in normal mice. Four proteins (FGF23, FGF23 A188, FGF23 A222 or FGF23 A188, 222 analogs) were each dosed to mice 0.5 mg/kg and 3 hours later quantitative real time PCR was employed to assess kidney mRNA expression of the FGFR/KL-downstream targets Cyp24a1, Cyp27b1 and early growth response protein 1 (Egr1). A significant elevation of Cyp24a1 along with coordinate suppression of Cyp27b1 was recorded for FGF23, FGF23 A188 and FGF23 A222 as compared to vehicle treatment (P<0.001, FIGS. 5B, 5C). For Egrl, FGF23 showed a statistically significant change as compared to vehicle (P<0.001), while single site mutants showed only a trend to activation (FIG. 5D). This is not surprising since the optimal treatment time for Egrl modulation is as early as 1 hour for FGF-signaling measurements. The magnitude in upregulation of gene expression for Cyp24a1 was the highest, with twenty-fold induction by native FGF23 treatment (FIG. 5B). For this particular target, it is clear that at the single administered dose of FGF23 A188 or FGF23 A222, the magnitude of the effect was fractionally reduced as compared to native FGF23 (P<0.01, FIG. 5B). Similarly, the Egrl induction is reduced for FGF23 A188 and FGF23 A222 analogs as compared to native FGF23 (P<0.05, FIG. 5D), but no difference is observed among the analogs and FGF23 for Cyp27b1 (FIG. 5C).

Of importance, the doubly mutated FGF23 A188,222 proved inactive relative to the native protein and the single site mutants (P<0.001 for Cyp24a1 and Cyp27b1, FIGS. 5B, 5C), and similar to the vehicle treated group. For Egrl, the FGF23 A188,222 was statistically different as compared to FGF23 treatment (P<0.01, FIG. 5D), and reduced when compared to the single site mutants but determined not to be statistically significant. Collectively, these results corroborate the existence of a novel second KL-site to support FGF23 bioactivity at molecular targets of physiological relevance. Furthermore, the differential Cyp24a1 activity (FIG. 5B) indicates that complete efficacy in biological signaling is only achieved when both KL-sites are functional.

The discovery of FGF23 as a phosphaturic hormone was a major advance in the physiology of phosphate metabolism. The discovery of the FGF23 gene mutations in ADHR patients was subsequently extended to other diseases including TIO, X-linked hypophosphatemia, and familial tumor calcinosis. Given its role in the regulation of bone-mineral metabolism the FGF23-pathway has emerged as a therapeutic target to address a range of bone-mineral and kidney disorders. The neutralizing FGF23 antibody Burosumab has reported efficacy in treatment of hypophosphatemia and is registered for treatment of XLH. The therapeutic use of the C-terminal peptide C72 has also been proposed for treatment of hypophosphatemia and renal anemia. The potential for broadened use of FGF-antagonism in treatment of chronic kidney disease remains exploratory as one study in rats reported improved overall disease symptoms, but it exacerbated mineral imbalance and was associated with higher mortality.

The C26 peptide is a competitive FGF23-antagonist, and its direct interaction with KL is supported by the structural data. This KL-binding site is critical for FGFR/KL-receptor activation and deletion of the remainder of the FGF23 C-terminus was previously reported as having consequence to cellular activity. This envisioned molecular mechanism of action in FGF23-signaling aligns with that of the related endocrine FGF-proteins FGF19 and FGF21. All three FGFs employ homologous linear C-terminal sequences of approximately twenty-five amino acids to bind a specific Klotho co-receptor. In each instance, the peptide as an independent fragment can competitively and selectively antagonize native protein signaling. Unlike FGF19 and FGF21 where the protein sequence terminates with the KLB-binding site, FGF23 is extended by an additional forty-six amino acids, representing a 20% increase in the full protein size. This raises the question as to whether this extended sequence is of any importance to biological function of the hormone, specifically as it relates to the FGF23-receptor complex interaction.

Within the extended portion of the FGF23 C-terminal previously suspected to have no biological function, a novel sequence (C28) was identified and it has partial homology to the known KL-binding site (C26) that can similarly function as a competitive peptide-based FGF23-antagonist (FIG. 1A, 1B, Table 1). Also, it has been shown by detailed structure activity analysis that the PRARM 195-199 sequence in C26 and the PMAS 218-221 sequence in C28, can be integrated into a chimeric peptide that demonstrates higher potency FGF23-antagonism than either of the native KL-interaction sites (FIG. 1C, 1D, Table 2). This structurally optimized antagonist (peptide 6) is of twenty-fold higher potency (IC₅₀ 3.7 nM) than the known KL-binding site (C26), and ten-fold enhanced relative to the novel second site (C28) identified. In each of the peptides C26 and C28, the site-specific mutation of an aspartic acid of known importance to KL-binding destroyed the peptide's bioactivity (FIG. 4A, Table 1). Extending these studies to the C72 peptide revealed a twenty-fold potency reduction when either of the two sites was individually inactivated (FIG. 4B, Table 1), but the peptides retained the activity corresponding to the shorter C26 and C28 peptides. Of note, when both sites are inactivated the FGF23-antagonism is lost (FIG. 4B, Table 1). These results collectively confirm the presence of a second functional sequence residing in the C-terminus of FGF23 that can regulate interactions in the FGFR receptor complex and significantly enhance the potency of KL-interaction in concert with the first site.

The length of the smallest functional KL-peptide antagonist was unexpectedly much shorter than what was anticipated based upon similar truncation in KLB-binding peptides and their ability to inhibit FGF19 or FGF21 agonism. A reduction of just two amino acids in these peptide antagonists of approximately twenty-five amino acids resulted in severe loss of KLB-dependent inhibition. A shortening to only twelve amino acids in the sequence optimized antagonist yielded peptide 12 with a potency that is equivalent to the native C26 peptide (FIG. 1F, Table 2). This observation indicates that there are striking differences in the requirements for high potency interactions at the two structurally related klotho co-receptors (KL and KLB), and suggests that the structural basis for FGF-agonism might not be as conserved as previously envisioned. Furthermore, in a pragmatic sense the reduction of the KL-mediated FGF23-inhibition to as little as twelve amino acids peptide represents a sizable advancement of therapeutic antagonists for treating bone-mineral diseases, where Burosumab is a hundred times larger in molecular size and represents a highly expensive medicine. The optimized antagonist peptide 6 when administered to mice, altered the renal gene expression of the vitamin D metabolizing enzymes Cyp24a1 and Cyp27b1 (FIG. 2), indicative of successful suppression of endogenous FGF23-mediated physiological targets.

Based upon the observation that C72 demonstrates higher activity than the individual peptides, dimeric forms of C26 and C28 were prepared (peptides 17, 18 and 19). These peptides were approximately twenty to fifty-fold more potent than their respective monomers, achieving a potency competitive with C72 (Table 2). The dimer of the optimized peptide 6, peptide 20 is slightly enhanced relative to the other peptide dimers and equal to that of C72 (Table 2). Native FGF23 attains optimal interaction with KL by utilizing its full C-terminus that is comprised of two sites that are individually suboptimal. A highly efficacious interaction was accomplished with non-native dimerization of monomeric peptides.

The individual mutation of Asp 188 or Asp 222 did not change FGF23 activity in 293/KL cells, while the double mutation destroyed FGF23-activity (FIG. 5A, Table 3). The results establish that each of the two sites is equally and independently capable of supporting FGF23 agonism, but unlike the study of antagonism in the C72 C-terminal peptide (FIG. 4B) there was no apparent additivity in the double-site functioning protein relative to the single site mutants. We hypothesized that this could potentially result from the highly optimized nature of the native FGF23 when studied in the engineered 293/KL cells that overexpress the KL co-receptor to achieve enhanced signal to noise, making it less amenable to observe finer downstream signaling changes. This prospect was explored through synthesis of a less optimal FGF23 mimetic where the N-terminal domain was provided by FGF21 and paired with one of two FGF23 C-terminal sequence extensions. A significant difference in the bioactivity was recorded for a FGF21-23 C72 chimeric protein that contains both KL-interaction sequences relative to a comparable but shorter chimeric protein FGF21-23 C26 which contains only a single KL-interaction site (Table 3). These results are supportive of the hypothesis that the benefit of having two sites relative to one is most evident when less than optimal interactions comprise the ternary-receptor complex.

To study the contribution of each KL-interaction site to native FGF23 function in a physiological setting, the FGF23-analogs were administered to normal mice and then their kidneys were examined as a representative target tissue. Native FGF23 provides a statistically significant change in the gene expression for the vitamin D metabolizing enzymes Cyp24a1 and Cyp27b1, as well as Egrl (FIGS. 5B-5D) (P<0.001) compared to vehicle treatment for each of the targets. The doubly mutated FGF23 A188,222 analog with inactivating mutations in both KL-sites was devoid of in vivo activity, consistent with the absence of in vitro activity. The FGF23 A¹⁸⁸ or FGF23 A²²² analogs where one of the two KL-sites is individually inactivated, demonstrated kidney target gene regulation directionally consistent with native FGF23 in a statistically significant manner (P<0.001) as compared to vehicle treatment for Cyp24a1 and Cyp27b1 (FIG. 5B-5C). This supports the conclusion that the novel second KL-site is functional, and the protein retains its activity in the absence of one of the two functioning KL-sites. Of note, the FGF3 A¹⁸⁸ or FGF23 A²²² analogs were less efficacious at inducing gene expression as compared to native FGF23 at a single equivalent dose of 0.5 mg/kg, most clearly seen in the instance of Cyp24a1 (FIG. 4b, P<0.01). Coupled with the absence of bioactivity of the double mutant it can be concluded that both sites are required to achieve full in vivo bioactivity. It is also possible that the pharmacokinetics of the native hormone is enhanced in total exposure or duration, despite comparable physical properties and the subtle nature of the structural change to the protein. Dose-titration and direct pharmacokinetic measures are required to determine whether there are differences in the specific in vivo potency of the single KL-site protein mutants. Nonetheless, the dovetailed in vitro and in vivo results presented here with peptide antagonists and protein agonists clearly establish the presence of a novel KL-site in the C-terminal fragment of FGF23 which was previously unrecognized.

It has been discovered that there is a second KL-site in native FGF23 capable of biological function and necessary for full FGF23 activity. The occurrence of this second FGF23 KL-site appears unique relative to FGF19 and FGF21 where signaling through the KLB co-receptor, occurs through a single KLB-binding site. Additionally, a highly potent peptide antagonist suitable for therapeutic use has been discovered, and it has been demonstrated that the minimal binding epitope for KL-antagonists is as small as twelve amino acids.

Acylated derivative of FGF23 peptide fragments

Acylated derivative of the peptide of SEQ ID NO 11 were prepared and tested for FGF23 antagonist activity.

Procedure for the synthesis of N-terminal fatty acid containing FGF23-inhibitor

Peptides were prepared as C-terminal amides by Fmoc solid-phase synthesis on Chem matrix Rink-amide resin. All the amino acid residues were incorporated to the peptide resin by an ABI-433A peptide synthesizer using a standard automated solid-phase coupling procedure with standard Fmoc-Oxyma-DCC 0.10 mmol methodology. The remaining unnatural amino acids, Fmoc-mini-Peg-OH, Fmoc-Glu-OtBu and C16 fatty acid (or C18 diacid) were coupled manually using DIC (1 mmol) and Oxyma (1 mmol) in DMF. The resin-cleavage was conducted in 10 mL of TFA solution containing 5% TIS and 1% H₂O at room temperature for two hours. The peptide was purified by reverse phase HPLC in a linear gradient of acetonitrile in aqueous 0.1% TFA.

Procedure for the synthesis of C-terminal fatty acid containing FGF23-inhibitor

Peptides were prepared as C-terminal amides by Fmoc solid-phase synthesis on Chem matrix Rink-amide. Fmoc-Lys(Mtt)-OH (1 mmol), DIC (1 mmol) and Oxyma (1 mmol) were dissolved in DMF (10 mL) and transferred to a reaction vessel for coupling to Chem matrix Rink-amide resin (0.1 mmol). The resulting resin mixture was gently agitated at room temperature for two hours, drained, washed with DMF (10 mL×3), followed by DCM (10 mL×3). The subsequent amino acid residues were coupled to the resulting resin (Fmoc-Lys(Mtt)-Chem matrix Rink amide) by an ABI-433A peptide synthesizer using a standard automated Fmoc-Oxyma-DCC 0.10 mmol methodology. The resulting resin was treated with 30% HFIP in DCM (10 mL) for 15 min twice to remove the Mtt group. The suspension was washed with DCM (10 mL×3) followed by DMF (10 mL×3). Fmoc-mini-Peg-OH, Fmoc-Glu-OtBu and C16 fatty acids (or C18 diacid) were sequentially coupled manually using DIC (lmmol) and Oxyma (1 mmol) in DMF. The peptide cleavage from the resin was conducted in 10 mL of TFA solution containing 5% TIS and 1% H₂O at room temperature for two hours. The peptide was purified by reverse phase HPLC in a linear gradient of acetonitrile in aqueous 0.1% TFA.

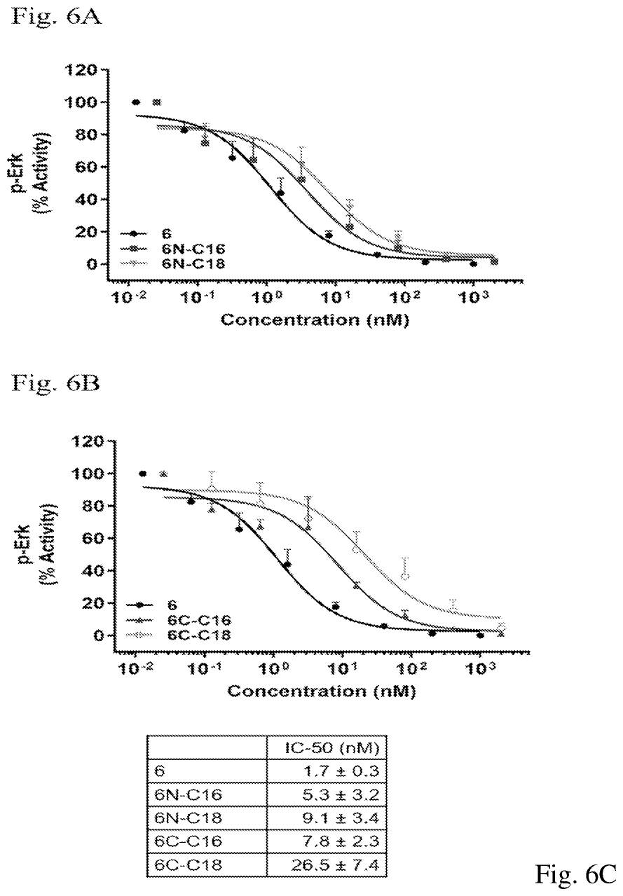

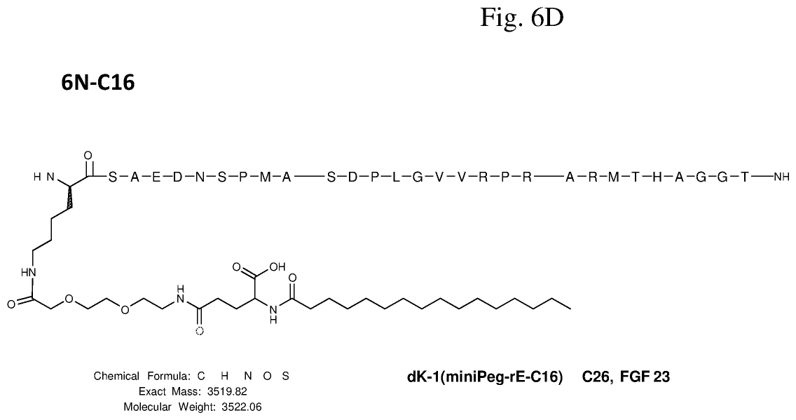

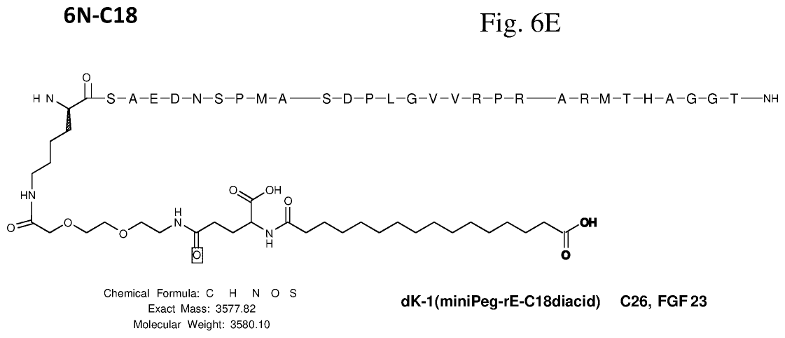

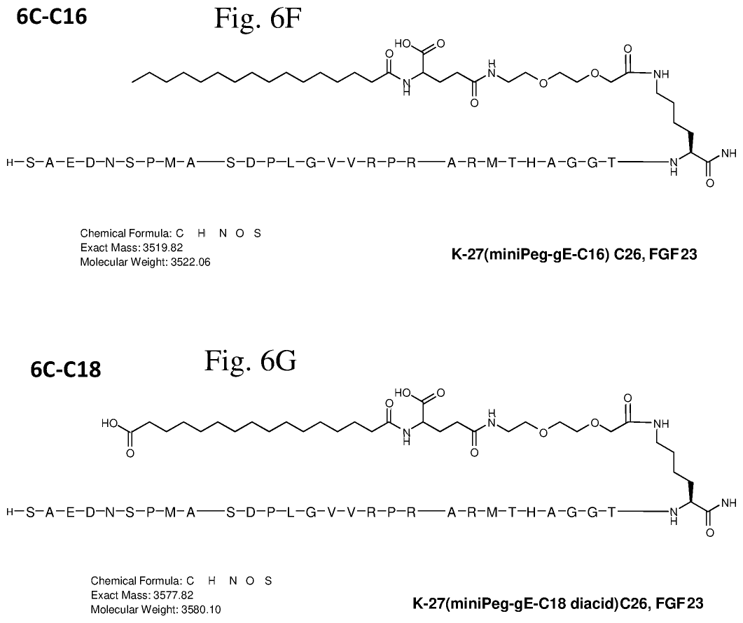

Results are shown in FIGS. 6A and 6B.

1. A peptide exhibiting antagonist activity against FGF23 binding to Klotho, said peptide comprising an amino acid sequence selected from the group consisting of SEQ ID NO: 15, SEQ ID NO: 16, SEQ ID NO: 17 or a peptide that differs from SEQ ID NO: 15, SEQ ID NO: 16, SEQ ID NO: 17 by one or two amino acid substitutions.

2. A peptide dimer comprising a first and second peptide independently selected from the peptides of claim 1, wherein the first and second peptides are covalently linked via a linker.

3. The peptide dimer of claim 2 wherein the dimer is a homodimer.

4. The peptide dimer of claim 2 wherein the dimer is a heterodimer.

5. The peptide dimer of claim 2 wherein the dimer is formed via a disulfide linkage between the side chain of an amino acid of each peptide.

6. The peptide dimer of claim 1 wherein said peptides comprising the dimer further comprises a C-terminal extension of 1 to 5 amino acids wherein the C-terminal amino acid of the extension is cysteine and a disulfide bond is formed between the side chains of said cysteine residues.

7. The peptide dimer of claim 1 wherein the linker is a peptide bond, an amino acid or a peptide that links the first and second peptides forming a linear contiguous amino acid chain.

8. The dimer of claim 7 comprising a homodimer sequence of compound 17-22 of Table 2.

9. The peptide of claim 1 wherein said peptide or dimer is fused to the carboxy terminus of the FGF23 core sequence, optionally wherein the FGF23 core sequence comprises a peptide of SEQ ID NO: 30 or a peptide that differs from SEQ ID NO: 30 by 1 to 3 amino acid substitutions.

10. A pharmaceutical composition comprising an FGF peptide of claim 1 and a pharmaceutically acceptable carrier, diluent, or excipient.

11. A method for reducing excessive actions of FGF23, comprising administering to a patient in need thereof a pharmaceutical composition of claim 10 in an amount effective to increase serum phosphate levels.

12. A method for treating a bone-mineral disease, said method comprising administering to a patient in need thereof a pharmaceutical composition of claim 10 in an amount effective to treat said disease.

13. The method of claim 12 wherein the disease is selected from the group consisting of hypophosphatemic rickets/osteomalacia including X-linked hypophosphatemic rickets (XLH) and tumor-induced osteomalacia.


SEQUENCE LISTING (30 sequences)

SEQ ID NO: 1 - protein, 26 aa, Artificial Sequence
  Synthetic construct
SAEDDSERDPLNVLKPRARMTPAPAS

SEQ ID NO: 2 - protein, 28 aa, Artificial Sequence
  Synthetic construct
SAEDNSPMASDPLGVVRGGRVNTHAGGT

SEQ ID NO: 3 - protein, 72 aa, Artificial Sequence
  Synthetic construct
SAEDDSERDPLNVLKPRARMTPAPASCSQELPSAEDNSPMASDPLGVVRGGRVNTHAGGTGPEGCRPFAKFI

SEQ ID NO: 4 - protein, 41 aa, Artificial Sequence
  Synthetic construct
PLIHFNTPIPRRHTRSAEDDSERDPLNVLKPRARMTPAPAS

SEQ ID NO: 5 - protein, 40 aa, Artificial Sequence
  Synthetic construct
SAEDNSPMASDPLGVVRGGRVNTHAGGTGPEGCRPFAKFI

SEQ ID NO: 6 - protein, 28 aa, Artificial Sequence
  Synthetic construct
SAEDDSPMASDPLNVLKPRARMTPAPAS

SEQ ID NO: 7 - protein, 26 aa, Artificial Sequence
  Synthetic construct
SAEDDSERDPLNVLKGGRVNTPAPAS

SEQ ID NO: 8 - protein, 26 aa, Artificial Sequence
  Synthetic construct
SAEDDSERDPLNVLKPRARMTHAGGT

SEQ ID NO: 9 - protein, 28 aa, Artificial Sequence
  Synthetic construct
SAEDDSERASDPLNVLKPRARMTPAPAS

SEQ ID NO: 10 - protein, 26 aa, Artificial Sequence
  Synthetic construct
SAEDNSERDPLGVVRGGRVNTHAGGT

SEQ ID NO: 11 - protein, 28 aa, Artificial Sequence
  Synthetic construct
SAEDNSPMASDPLGVVRPRARMTHAGGT

SEQ ID NO: 12 - protein, 28 aa, Artificial Sequence
  Synthetic construct
SAEDNSPMASDPLGVVRGGRVNTPAPAS

SEQ ID NO: 13 - protein, 14 aa, Artificial Sequence
  Synthetic construct
ERDPLNVLKPRARM

SEQ ID NO: 14 - protein, 12 aa, Artificial Sequence
  Synthetic construct
DPLNVLKPRARM

SEQ ID NO: 15 - protein, 16 aa, Artificial Sequence
  Synthetic construct
PMASDPLGVVRPRARM

SEQ ID NO: 16 - protein, 14 aa, Artificial Sequence
  Synthetic construct
ASDPLGVVRPRARM

SEQ ID NO: 17 - protein, 12 aa, Artificial Sequence
  Synthetic construct
DPLGVVRPRARM

SEQ ID NO: 18 - protein, 31 aa, Artificial Sequence
  Synthetic construct
SAEDDSERDPLNVLKPRARMTPAPASGPEGC

SEQ ID NO: 19 - protein, 33 aa, Artificial Sequence
  Synthetic construct
SAEDNSPMASDPLGVVRGGRVNTHAGGTGPEGC

SEQ ID NO: 20 - protein, 33 aa, Artificial Sequence
  Synthetic construct
SAEDNSPMASDPLGVVRPRARMTHAGGTGPEGC

SEQ ID NO: 21 - protein, 33 aa, Artificial Sequence
  Synthetic construct
SAEDNSPMASAPLGVVRPRARMTHAGGTGPEGC

SEQ ID NO: 22 - protein, 26 aa, Artificial Sequence
  Synthetic construct
SAEDDSERAPLNVLKPRARMTPAPAS

SEQ ID NO: 23 - protein, 26 aa, Artificial Sequence
  Synthetic construct
SAEDDSERAPLNVLKPRARMTPAPAS

SEQ ID NO: 24 - protein, 72 aa, Artificial Sequence
  Synthetic construct
SAEDDSERAPLNVLKPRARMTPAPASCSQELPSAEDNSPMASDPLGVVRGGRVNTHAGGTGPEGCRPFAKFI

SEQ ID NO: 25 - protein, 72 aa, Artificial Sequence
  Synthetic construct
SAEDDSERDPLNVLKPRARMTPAPASCSQELPSAEDNSPMASAPLGVVRGGRVNTHAGGTGPEGCRPFAKFI

SEQ ID NO: 26 - protein, 72 aa, Artificial Sequence
  Synthetic construct
SAEDDSERAPLNVLKPRARMTPAPASCSQELPSAEDNSPMASAPLGVVRGGRVNTHAGGTGPEGCRPFAKFI

SEQ ID NO: 27 - protein, 26 aa, Artificial Sequence
  Synthetic construct
PLETDSMDPFGLVTGLEAVRSPSFEK

SEQ ID NO: 28 - protein, 25 aa, Artificial Sequence
  Synthetic construct
PPDVGSSDPLSMVGPSQGRSPSYAS

SEQ ID NO: 29 - protein, 251 aa, Artificial Sequence
  synthetic construct
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 30 - protein, 155 aa, Artificial Sequence
  Synthetic construct
YPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTR